(19) United States
(12) Patent Application Publication
(10) Pub. No.: US 2009/0317375 A1
(43) Pub. Date: Dec. 24, 2009

(54) VON WILLEBRAND FACTOR (VWF) INHIBITORS FOR TREATMENT OR PREVENTION OF INFARCTION

(75) Inventors: Denisa Wagner, Dover, MA (US); Bing-Qiao Zhao, Beijing (CN)

Correspondence Address: BAXTER HEALTHCARE CORPORATION, ONE BAXTER PARKWAY, MAIL STOP DF2-2E, DEERFIELD, IL, 60015, US

(73) Assignee: Immune Disease Institute, Inc., Boston, MA (US)

(21) Appl. No.: 12/437,384

(22) Filed: May 7, 2009

Related U.S. Application Data
(60) Provisional application No. 61/127,426, filed on May 12, 2008.

(51) Int. Cl. A61K 38/48

(52) U.S. Cl. 424/94.67

(57) ABSTRACT

This invention relates to methods for treating or preventing an infarction by administering to a patient in need thereof a compound capable of suppressing the expression or activity of the von Willebrand Factor (VWF). Thus, the invention relates to the use of a pharmaceutically effective amount of a VWF inhibitor, such as ADAMTS13, for the preparation of a medicament for treating conditions known to involve infarction to reduce or eliminate the symptoms and effect of an infarction.

### CROSS REFERENCE TO RELATED PATENT APPLICATIONS

The present patent application claims benefit to U.S. Provisional Patent Application 61/127,426, filed May 12, 2008, which is incorporated by reference in its entirety.

### FIELD OF THE INVENTION

This invention relates to methods of treating or preventing infarction by administration of an effective amount of an inhibitor of the von Willebrand Factor (VWF), such as ADAMTS13, in a patient in need thereof. Thus, the invention permits the use of a VWF inhibitor for the preparation of a pharmaceutical composition for reducing or preventing infarction in a patient who is suffering/has suffered from a condition that can lead to infarction or is at risk of such a condition.

### BACKGROUND OF THE INVENTION

An infarction is the process resulting in a macroscopic area of necrotic tissue in an organ caused by loss of adequate blood supply. Supplying arteries can be blocked from within by some obstruction (e.g., a blood clot or fatty cholesterol deposit), or can be mechanically compressed or ruptured by trauma. Infarctions are commonly associated with atherosclerosis, where an atherosclerotic plaque ruptures, a thrombus forms on the surface occluding the blood flow and occasionally forming an embolus that occludes other blood vessels downstream. Infarctions in some cases involve mechanical blockage of the blood supply, such as when part of the gut herniates or twists.

Infarctions can be generally divided into two types according the amount of hemorrhaging present: one type is anemic infarction, which affects solid organs such as the heart, spleen, and kidneys. The occlusion is most often composed of platelets, and the organ becomes white, or pale. The second is hemorrhagic infarctions, affecting, e.g, the lungs, brain, etc. The occlusion consists more of red blood cells and fibrin strands.

Diseases commonly associated with infarctions include: myocardial infarction (heart attack), pulmonary embolism, cerebrovascular events such as stroke, peripheral artery occlusive disease (such as gangrene), antiphospholipid syndrome, sepsis, giant-cell arteritis (GCA), hernia, and volvulus.

Because of the serious and irreversible nature of infarctions, there exists a clear need for new and effective methods to reduce the level and extent of an infarction or to prevent the occurrence of an infarction. The present invention addresses this need while reducing the likelihood of side effects observed with existing therapies.

### BRIEF SUMMARY OF THE INVENTION

The present invention relates to a method for treating or preventing an infarction in an individual (patient), comprising the step of administering to the individual a pharmaceutical composition comprising a VWF inhibitor in an amount that is effective to suppress the expression or activity of VWF. In some embodiments, the inhibitor is ADAMTS13 protein or a biologically active derivative there of. The biologically active derivative is a chimeric molecule can comprise ADAMTS13 or a biologically active derivative thereof and a heterologous protein, e.g., an immunoglobulin or a biologically active derivative thereof. In some embodiments, the VWF inhibitor reduces the ability of VWF to form high molecular weight multimers, promote infarction, or promote blood clotting.

In some embodiments, the infarction is in the brain, heart, or lung. In some embodiments, the ADAMTS13 protein or biologically active derivative thereof is administered at a dose of 10-10,000 U/kg body weight of the individual. In some embodiments, dose is about 100, 500, 1000, 2000, 3000, 3258, or 5000 U/kg body weight of the individual. In some embodiments, the level of plasma VWF, particularly UL-VWF, is determined before determining the dose of ADAMTS13 protein. In some embodiments, the dose of ADAMTS13 protein or biologically active derivative thereof is based on the plasma level of VWF, particularly UL-VWF, in the individual.

In some embodiments, the method comprising the step of administering an additional active ingredient, which is selected from the group consisting of agents that stimulate ADAMTS13 production/secretion; agents that inhibit ADAMTS13 degradation; agents that enhance ADAMTS13 activity; and agents that inhibit ADAMTS13 clearance from circulation. In some embodiments, the inhibitor is an inactivating VWF antibody.

In some embodiments, the ADAMTS13 or derivative thereof is recombinantly produced, e.g., by HEK293 cells or CHO cells. In some embodiments, the ADAMTS13 protein or derivative thereof is glycosylated, e.g., in the same pattern as that produced in CHO cells. In some embodiments, the ADAMTS13 or derivative thereof is glycosylated in the same pattern as that produced in HEK293 cells. In some embodiments, the ADAMTS13 or derivative thereof has a plasma half-life of at least one hour, e.g., 2, 3, 4, 5, 6, or more hours.

In some embodiments, the pharmaceutical composition is administered more than once, e.g., to an individual with a chronic condition, high risk of infarction (e.g., genetic), or to prevent recurrence of infarction. In some embodiments, the pharmaceutical composition is administered by continuous infusion. In some embodiments, the pharmaceutical composition is administered immediately upon discovery of the infarction, e.g., within 15, 30, 60, 90, 110, 120 minutes. However the pharmaceutical composition can still be beneficial if administered at a later time post-infarction (e.g., more than 6 hours or several days).

In some embodiments, said administration reduces infarct volume 22 hours after administration. In some embodiments, said administration does not significantly affect a peripheral immune response, e.g., as compared to the immune response in an individual or population of individuals not receiving treatment. In some embodiments, said administration does not increase the level of hemorrhage in the individual, e.g., as compared to the level of hemorrhage in an individual or population of individuals not receiving treatment. In some cases, the likelihood of peripheral immune response and/or hemorrhage increases post-infarction.

The invention further provides methods of reducing the harmful side effects of infarction, in particular, cerebral infarction. In some embodiments, the invention provides a method of improving the recovery of (or reducing the damage to) sensory and/or motor function in an individual after a cerebral infarction, comprising the step of administering to the individual a pharmaceutical composition comprising a therapeutically effective amount of an ADAMTS13 protein or a biologically active derivative thereof, thereby improving the recovery of (or reducing the damage to) sensory and/or motor function in the individual post-cerebral infarction. In some embodiments, the pharmaceutical composition is administered immediately upon discovery of the cerebral infarction, e.g., within 15, 30, 60, 90, 110, 120 minutes. In some embodiments, the ADAMTS13 protein or a biologically active derivative thereof is administered at a dose of 10-10,000 U/kg body weight of the individual. In some embodiments, dose is about 100, 500, 1000, 2000, 3000, 3258, or 5000 U/kg body weight of the individual.

The invention provides the use of a pharmaceutically effective amount of a VWF inhibitor for the manufacture or preparation of a pharmaceutical composition for treating or preventing an infarction. In some embodiments, the inhibitor is ADAMTS13 protein or a biologically active derivative thereof. For example, a biologically active derivative can be a chimeric molecule comprising ADAMTS13 or a biologically active derivative thereof and an immunoglobulin or a biologically active derivative thereof. The ADAMTS13 protein be recombinantly produced by, e.g., HEK293 cells or CHO cells.

In some embodiments, the ADAMTS13 protein or its biologically active derivative is combined with an additional active ingredient, which is selected from the group consisting of: blood thinning agents; agents that stimulate ADAMTS13 production/secretion; agents that inhibit ADAMTS13 degradation; agents that enhance ADAMTS13 activity; and agents that inhibit ADAMTS13 clearance from circulation. In some embodiments, the ADAMTS13 protein or derivative thereof is glycosylated, e.g., in the same pattern as that produced in CHO cells. In some embodiments, the ADAMTS13 or derivative thereof is glycosylated in the same pattern as that produced in HEK293 cells. In some embodiments, the ADAMTS13 or derivative thereof has a plasma half-life of at least one hour, e.g., 2, 3, 4, 5, 6, or more hours.

### BRIEF DESCRIPTION OF THE DRAWINGS

FIG. 1. Deficiency in VWF reduces infarct volume in the intraluminal MCAO model in mice. Transient occlusion of the right middle cerebral artery (MCA) was achieved by a monofilament insertion up to the MCA following standard procedures. After 2 hours, the monofilament was withdrawn to allow reperfusion. Infarct volume was measured by 2% 2,3,5-triphenyltetrazolium hydrochloride (TTC) staining at 24 h after cerebral ischemia. Data are expressed as mean±SEM (n=10).

FIG. 2. Level of VWF regulates infarct volume after ischemic stroke in mice. Representative TTC stain of coronal brain sections of one mouse for each strain 22 h after MCAO (top) and brain infarct volumes (bottom) in WT, Vwf± and Vwf−/− mice. Deficiency or heterozygosity of VWF resulted in a significant decrease in infarct volume compared to WT.

FIG. 3. Recombinant human VWF increases infarct volume. Mice were subjected to 2 h transient focal ischemia. Recombinant human VWF (0.8 mg/kg body weight) was infused 10 min before reperfusion and repeated 3 h later. Treatment with rhVWF increased infarct volume 24 h after stroke compared with vehicle-treated control group. Data are expressed as mean±SEM (n=4-5).

FIG. 4. Deficiency of ADAMTS13 (ATS13−/−) increases infarct volume. Mice were subjected to 2 h transient focal ischemia and infarct volume was measured 24 h after stroke. Data are expressed as mean±SEM (n=13-15).

FIG. 5. Level of ADAMTS13 regulates infarct volume after ischemic stroke in mice. Representative TTC stain of coronal brain sections of one mouse for each strain 22 h after focal cerebral ischemia in WT, Adamts13−/− and Adamts13−/−/Vwf−/− (top) and corresponding brain infarct volumes quantification (bottom). Increase in infarct volume in Adamts13−/− mice, when compared to WT, was dependent on the presence of VWF.

FIG. 6. Recombinant human ADAMTS13 (rhATS13) reduces infarct volume. Mice were subjected to 2 h transient focal ischemia and infarct volume was measured 24 h after stroke. Recombinant human ADAMTS13 (3258 U/kg body weight) was infused 10 min before reperfusion. Compared with the vehicle-treated group, administration of rhADAMTS13 derived from HEK293 cells significantly reduced infarct volume (n=9). Treatment with rhADAMTS13 derived from CHO cells also resulted in a reduction in infarct volume. Data are expressed as mean±SEM (n=4).

FIG. 7. Recombinant human ADAMTS13 reduces infarct volume after focal cerebral ischemia in WT mice. Representative TTC staining of coronal brain sections of one mouse for each treatment and infarct volumes 22 h after focal cerebral ischemia in mice treated with (A) vehicle or r-hu ADAMTS13 (HEK 293 cells derived) and (B), vehicle or r-hu ADAMTS13 (CHO cells derived) are shown.

FIG. 8. Recombinant human ADAMTS13 improves performances in the tape removal test after ischemic stroke. Time to remove the contralateral (A) and ipsilateral (B) adhesive tapes were recorded on sham-operated mice and MCAO mice injected intravenously with r-hu ADAMTS13 or vehicle 10 min before reperfusion. Global differences between groups were found for each parameter (p<0.05).

FIG. 9. Effect of the r-hu ADAMTS13 preparations on cerebral hemorrhage and tail bleeding time. (A) Representative unstained coronal brain sections of one mouse for each treatment show a lack of hemorrhage in r-hu ADAMTS13-treated mice (HEK and CHO cells derived). (B) Bleeding time measurements show highly increased bleeding in Vwf−/− mice compared with WT. All the Vwf−/− mice were cauterized at 900 sec to stop bleeding. r-hu ADAMTS13-treated mice (5 h) had a bleeding time comparable to WT (prepared in HEK cells) or prolonged bleeding time (prepared in CHO cells) but significantly shorter than the Vwf−/− mice. n=8 each group.

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It can include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids can be referred to herein by either the commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N.Y. (1984)).

In the present application, amino acid residues are numbered according to their relative positions from the left most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a core amino acid sequence responsible for NRG-integrin binding has at least 80% identity, preferably 85%, 90%, 91%, 92%, 93, 94%, 95%, 96%, 97%, 98%, 99%, or 100% identity, to a reference sequence, e.g., SEQ ID NO:1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments can be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites can be wild type or modified by one or more amino acid substitutions, e.g., modified to resemble human immunoglobulin more closely. Both chimeric and humanized antibodies are made using recombinant techniques, which are well-known in the art (see, e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody).

“Modulators” of activity are used to refer to ligands, antagonists, inhibitors, activators, and agonists, e.g., identified using in vitro and in vivo assays for activity, e.g., thrombolytic activity. Modulators can be naturally occurring, a mimetic based on a naturally occurring ligand, or synthetic. Assays to identify, e.g., a VWF antagonist or agonist include, e.g., applying putative modulator compounds to cells or an animal model, in the presence or absence of VWF and then determining the functional effects on VWF activity. Samples or assays comprising VWF that are treated with potential modulators are compared to control samples without the modulators to examine the extent of effect. Control samples (untreated with modulators) are assigned a relative activity value of 100%.

The terms “inhibiting (inhibition),” antagonizing (antagonism),” “reducing (reduction),” or “suppressing (suppression),” as used herein, refer to any detectable negative effect on a target biological activity or process, such as the activity of von Willebrand Factor, or the volume of infarct resulted from a disease or condition. Typically, an inhibition is reflected in a decrease of at least 10%, 20%, 30%, 40%, or 50% in infarct volume, when compared to a control. An “inhibitor” is a compound capable of inhibiting a target activity or process.

The terms “VWF inhibitor” or “VWF antagonist” are used interchangeably herein. A VWF inhibitor is an agent that reduces the ability of VWF to participate in blood clotting, form large multimers, promote thrombosis, promote infarction, etc. VWF inhibitors also include agents that promote bleeding/ reduce clotting. Inhibition is achieved when at least one VWF activity relative to a control is significantly reduced (e.g., with reference to a desired statistical measure), as can be determined by one of skill in the art. Generally, activity of about 80%, 70%, 60%, 50%, or 25-1% of the control activity indicates the presence of an inhibitor.

The terms “VWF activator” or “VWF agonist” are used interchangeably herein. Activation is achieved when at least one VWF activity (e.g., clotting, thrombogenesis) relative to a control is significantly increased (e.g., with reference to a desired statistical measure), as can be determined by one of skill in the art. Generally, activity of about 110%, 125%, 150%, 200%, 300%, 500%, or 1000% or more of the control activity indicates the presence of an agonist.

The terms “inhibit” or “activate” or “modulate,” when referring to expression or activity, are not intended as absolute terms. For example, if an agent “does not inhibit” or “does not activate” a given polypeptide, it generally means that the agent does not have a statistically significant effect on the polypeptide, e.g., as compared to a control or range of controls. The terms “reduce” and “increase” and similar relative terms are used herein to refer to a reductions, increases, etc. relative to a control value. Those of skill in the art are capable of determining an appropriate control for each situation. For example, if an agent is said to “reduce binding” of X to Y, the level of X-Y binding in the presence of the agent is reduced compared to the level of X-Y binding in the absence of the agent.

The term “effective amount,” as used herein, refers to an amount that produces therapeutic effects for which a substance is administered. The effects include the prevention, correction, or inhibition of progression of the symptoms of a disease/condition (such as infarction) and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the terms “treat” and “prevent” are not intended to be absolute terms. Treatment can refer to any delay in onset, amelioration of symptoms, improvement in patient survival, reduction of infarct volume, reduction in frequency or severity, etc. Thus, the term “treatment” can include prevention. The effect of treatment can be compared to a control, e.g., an individual or pool of individuals not receiving the treatment, an untreated tissue in the same patient, or the same individual prior to treatment.

A “biological sample” can be obtained from a patient, e.g., a biopsy, from an animal, such as an animal model, or from cultured cells, e.g., a cell line or cells removed from a patient and grown in culture for observation. Biological samples include tissue such as colorectal tissue or bodily fluids, e.g., blood, blood fractions, lymph, saliva, urine, feces, etc.

### DETAILED DESCRIPTION OF THE INVENTION

### I. Introduction

Ischemic events, such as heart attack and stroke, are a leading cause of death and disability around the world. Thrombolytic therapy with tissue plasminogen activator (tPA), which leads to fibrin degradation and promotes clot lysis, can be used to treat ischemia, but tPA use is restricted to the first few hours after the ischemic event. In addition, tPA can increase incidence and severity of hemorrhage and edema formation. Thus, there remains a clear need to identify new therapeutic agents for minimizing the effects of ischemia. In addition to its effect on coagulation, such agents can also target platelet adhesion and the inflammatory process that follows ischemic events.

von Willebrand Factor (VWF) is a large multimeric glycoprotein that is present in blood plasma and plays a major role in blood coagulation. VWF is stored in an ultra large form (UL-VWF, >20 million Da) in platelet a-granules and Weibel-Palade bodies of endothelial cells from which it is released during injury or inflammation. If not immediately consumed for platelet adhesion, the UL-VWF is cleaved by ADAMTS13 to smaller less adhesive multimers that circulate in plasma. Ischemia, such as occurs after thrombolysis, is a potent inducer of Weibel-Palade body secretion, thus making the infarct area highly thrombogenic.

The basic VWF monomer is a 2050-amino acid protein that includes a number of specific domains with a specific function: (1) the D′/D3 domain, which binds to Factor VIII; (2) the A1 domain, which binds to platelet GP1b-receptor, heparin, and possibly collagen; (3) the A3 domain, which binds to collagen; (4) the C1 domain, in which the R-G-D motif binds to platelet integrin αIIbβ3 when this is activated; and (5) the “cysteine knot” domain located at the C-terminus, which VWF shares with platelet-derived growth factor (PDGF), transforming growth factor-β (TGFβ), and β-human chorionic gonadotropin (βHCG).

Multimers of VWF can be extremely large, consisting of over 80 monomers with molecular weight exceeding 20,000 kDa. These large VWF multimers are most biologically functional, capable of mediating the adhesion of platelets to sites of vascular injury, as well as binding and stabilizing the procoagulant protein Factor VIII. Deficiency in VWF or altered VWF is known to cause various bleeding disorders.

The biological breakdown of VWF is largely mediated by a protein termed ADAMTS13 (A Disintegrin-like And Metalloprotease with Thrombospondin type I motif No. 13), a 190 kDa glycosylated protein produced predominantly by the liver. ADAMTS13 is a plasma metalloprotease that cleaves VWF between tyrosine at position 1605 and methionine at position 1606, breaking down the VWF multimers into smaller units, which are further degraded by other peptidases.

The present inventors discovered that VWF plays a role in infarction, a process in which tissue undergoes necrosis due to insufficient blood supply. The inventors' studies showed that, when VWF level is suppressed, infarct volume is reduced; whereas increased level of VWF leads to larger infarct volume. More specifically, the inventors are able to demonstrate that ADAMTS13, the enzyme that cleaves and reduces VWF activity, can be used to reduce or limit the volume of infarct.

In particular, the inventors have uncovered a crucial role for the VWF-ADAMTS13 axis in regulating ischemic stroke. Both VWF level and its thrombotic activity, as reflected by multimer size, impact heavily on stroke outcome. ADAMTS13 provides a significant protective effect by reducing final infarct volume without increasing the likelihood of hemorrhage. Measurement of VWF and ADAMTS13 levels can be used to indicate the likelihood of transient ischemic attacks and stroke in humans. Importantly, infusion of r-hu ADAMTS13 into WT mice reduced infarct size and significantly improved functional outcome without inducing cerebral hemorrhage. Pharmaceutical preparations based on ADAMTS13 and ADAMTS13 derivatives offer a new safer option for treatment of ischemic stroke.

### II. Use of VWF Inhibitors to Treat Infarction

One aspect of the present invention relates to a method of reducing the volume of infarct or inhibiting infarct from forming by administering to a patient in need thereof (e.g., a person having or at risk of having a condition that can lead to infarction) an effective amount of an inhibitor of von Willebrand Factor (VWF). Such an inhibitor can be any compound capable of suppressing the production of VWF or the activity of VWF. Some examples of VWF inhibitors include ADAMTS13 or its biologically active derivatives, inactivating antibodies of VWF, siRNA that can inhibit VWF synthesis, and various small molecules.

### A. ADAMTS13

The term “biologically active derivative” as used herein means any polypeptides with substantially the same biological function as ADAMTS13, particularly in its ability. The polypeptide sequences of the biologically active derivatives can comprise deletions, additions and/or substitution of one or more amino acids whose absence, presence and/or substitution, respectively, do not have any substantial negative impact on the biological activity of polypeptide. The biological activity of said polypeptides can be measured, for example, by the reduction or delay of platelet adhesion to the endothelium or subendothelium, the reduction or delay of platelet aggregation in a flow chamber, the reduction or delay of the formation of platelet strings, the reduction or delay of thrombus formation, the reduction or delay of thrombus growth, the reduction or delay of vessel occlusion, the proteolytical cleavage of VWF, and/or the reduction of infarct volume in an experimental system similar to those described in the Examples Section of this application.

The terms “ADAMTS13” and “biologically active derivative”, respectively, also include polypeptides obtained via recombinant DNA technology. Recombinant ADAMTS13 (“rADAMTS13”), e.g., recombinant human ADAMTS13 (“r-hu-ADAMTS13”), can be produced by any method known in the art. One specific example is disclosed in WO 02/42441 with respect to the method of producing recombinant ADAMTS13. This can include any method known in the art for (i) the production of recombinant DNA by genetic engineering, e.g., via reverse transcription of RNA and/or amplification of DNA, (ii) introducing recombinant DNA into prokaryotic or eukaryotic cells by transfection, i.e., via electroporation or microinjection, (iii) cultivating said transformed cells, e.g., in a continous or batchwise manner, (iv) expressing ADAMTS13, e.g., constitutively or upon induction, and (v) isolating said ADAMTS13, e.g., from the culture medium or by harvesting the transformed cells, in order to (vi) obtain substantially purified recombinant ADAMTS13, e.g., via anion exchange chromatography or affinity chromatography. The term “biologically active derivative” includes also chimeric molecules such as ADAMTS13 (or a biologically active derivative thereof) in combination with an immunoglobulin molecule (Ig), in order to improve the biological/pharmacological properties such as half life of ADAMTS13 in the circulation system of a mammal, particularly human. The Ig could have also the site of binding to an Fc receptor optionally mutated.

The rADAMTS13 can be produced by expression in a suitable prokaryotic or eukaryotic host system characterized by producing a pharmacologically effective ADAMTS13 molecule. Examples of eukaryotic cells are mammalian cells, such as CHO, COS, HEK 293, BHK, SK-Hep, and HepG2. There is no particular limitation to the reagents or conditions used for producing or isolating ADAMTS13 according to the present invention and any system known in the art or commercially available can be employed. In one embodiment of the present invention rADAMTS13 is obtained by methods as described in the state of the art.

A wide variety of vectors can be used for the preparation of the rADAMTS13 and can be selected from eukaryotic and prokaryotic expression vectors. Examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOX1, GAP, GAL1, AUG1, etc; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived form viral systems such as vaccinia virus, adeno-associated viruses, herpes viruses, retroviruses, etc., using promoters such as CMV, SV40, EF-1, UbC, RSV, ADV, BPV, and β-actin.

### B. Pharmaceutical Compositions

The invention provides pharmaceutical compositions useful for reducing the volume of infarct or inhibiting infarct from forming in a patient. Such a composition comprises an effective amount of an inhibitor of von Willebrand Factor (VWF), which can be any compound capable of suppressing the production of VWF or the activity of VWF. One example is ADAMTS13 or its biologically active derivatives. The invention thus provides a novel use of a VWF inhibitor for the preparation or manufacture of a medicament to treating or preventing infarction, which is frequently associated with serious conditions such as cardiovascular, pulmonary, and cerebrovascular emergencies.

The pharmaceutical composition of the invention can comprise one or more pharmaceutically acceptable carrier and/or diluent. The pharmaceutical composition can also comprise one or more additional active ingredients such as agents that stimulate ADAMTS13 production or secretion by the treated patient/individual, agents that inhibit the degradation of ADAMTS13 and thus prolong its half life (or alternatively glycosylated variants of ADAMTS13), agents that enhance ADAMTS13 activity (for example by binding to ADAMTS13, thereby inducing an activating conformational change), or agents that inhibit ADAMTS13 clearance from circulation, thereby increasing its plasma concentration.

As VWF levels vary widely between individuals, the dosage of ADAMTS13 can be determined on an individual basis, as best determined by a medical professional. The pharmaceutically effective amount of ADAMTS13 or a biologically active derivative thereof can range, for example, from 0.1 to 20 mg/kg body weight. In some embodiments, the amount of ADAMTS13 administered is based on U activity. Exemplary dosages include 10 U-10,000 U/kg body weight. For example, ADAMTS13 or a biologically active derivative of ADAMTS13 can be administered at 10, 50, 100, 200, 500, 1000, 2000, 3000, 3500, 5000, 6000, 7000, 8000, or 10,000 U/kg body weight, and the dose can optionally be determined based on individual plasma VWF levels. Dose can also be determined based on whether the ADAMTS13 is administered prophylatically (e.g., in a repeated doses) or in response to a medical emergency, to immediately reduce harmful effects of an infarction.

It must be kept in mind that the compositions of the present invention can be employed in serious disease states, that is, life-threatening or potentially life threatening situations. In such cases, in view of the lack of side effects (e.g., hemorrhage, immune system effects), it is possible and may be felt desirable by the treating physician to administer substantial excesses of the pharmaceutical compositions of the invention.

ADAMTS13 or its biologically active derivative can be administered with one or more additional active ingredients such as agents that stimulate ADAMTS13 production or secretion by the treated patient/individual, agents that inhibit the degradation of ADAMTS13 and thus prolonging its half life, agents that enhance ADAMTS13 activity (for example by binding to ADAMTS13, thereby inducing an activating conformational change), or agents that inhibit ADAMTS13 clearance from circulation, thereby increasing its plasma concentration. Another ingredient that can be co-administered include blood thinners (e.g., aspirin), anti-platelet agents, and tissue plasminogen activator (tPA), a serine protease that activates plasmin to cleave fibrin.

The route of administration does not exhibit a specific limitation and can be, for example, subcutaneous or intravenous. Oral administration of VWF inhibitors is also a possibility. The term “patient” as used in the present invention includes mammals, particularly human.

The VWF inhibitors of the present invention can be administered to mammals, particularly humans, for prophylactic and/or therapeutic purposes. In some embodiments, the present invention is used to reduce the harmful effects of infarction, without increasing the likelihood of hemorrhage or disabling the peripheral immune system. In some embodiments, the VWF inhibitors are administered prophylactically, e.g., to an individual at risk of infarction. In such cases, prophylactic treatment is usually repeated at a lower dose for an extended period of time, e.g., for a given period of time after an initial infarction event. Examples of individuals that can be treated according to the invention include those that have experienced an infarction, such as a heart attack, a pulmonary infarction, or stroke, no matter the severity. This is especially true if the VWF inhibitor can be administered soon after the infarction, to reduce the tissue damage that results from loss of blood to the surrounding tissues. VWF inhibitors can be administered to individuals at risk of experiencing infarction, e.g., as a result of illness or blood pressure related condition, surgery, or other medication.

Therapeutic administration can begin at the first sign of infarction or shortly after diagnosis, e.g., to prevent recurrence. This can be followed by boosting doses for a period thereafter. In chronically affected individuals, long term treatment can be provided.

### C. Other VWF Inhibitors

Inhibitory Nucleic Acids

Inhibition of VWF expression can be achieved through the use of inhibitory nucleic acids. Inhibitory nucleic acids can be single-stranded nucleic acids or oligonucleotides that can specifically bind to a complementary nucleic acid sequence. By binding to the appropriate target sequence, an RNA-RNA, a DNA-DNA, or RNA-DNA duplex or triplex is formed. These nucleic acids are often termed “antisense” because they are usually complementary to the sense or coding strand of the gene, although recently approaches for use of “sense” nucleic acids have also been developed. The term “inhibitory nucleic acids” as used herein, refers to both “sense” and “antisense” nucleic acids.

In one embodiment, the inhibitory nucleic acid can specifically bind to a target VWF polynucleotide. Administration of such inhibitory nucleic acids can reduce or inhibit infarction by reducing or eliminating the effects of VWF in a patient. Nucleotide sequences encoding VWF are known for several species, including the human cDNA sequence. One can derive a suitable inhibitory nucleic acid from the human VWF, species homologs, and variants of these sequences.

By binding to the target nucleic acid, the inhibitory nucleic acid can inhibit the function of the target nucleic acid. This could, for example, be a result of blocking DNA transcription, processing or poly(A) addition to mRNA, DNA replication, translation, or promoting inhibitory mechanisms of the cells, such as promoting RNA degradation. Inhibitory nucleic acid methods therefore encompass a number of different approaches to altering expression of specific genes that operate by different mechanisms. These different types of inhibitory nucleic acid technology are described in Helene and Toulme (1990) Biochim. Biophys. Acta., 1049:99-125.

The inhibitory nucleic acids introduced into the cell can also encompass the “sense” strand of the gene or mRNA to trap or compete for the enzymes or binding proteins involved in mRNA translation. See Helene and Toulme, supra.

The inhibitory nucleic acids can also be used to induce chemical inactivation or cleavage of the target genes or mRNA. Chemical inactivation can occur by the induction of crosslinks between the inhibitory nucleic acid and the target nucleic acid within the cell. Alternatively, irreversible photochemical reactions can be induced in the target nucleic acid by means of a photoactive group attached to the inhibitory nucleic acid. Other chemical modifications of the target nucleic acids induced by appropriately derivatized inhibitory nucleic acids can also be used.

Cleavage, and therefore inactivation, of the target nucleic acids can be effected by attaching to the inhibitory nucleic acid a substituent that can be activated to induce cleavage reactions. The substituent can be one that effects either chemical, photochemical or enzymatic cleavage. For example, one can contact an mRNA:antisense oligonucleotide hybrid with a nuclease which digests mRNA:DNA hybrids. Alternatively cleavage can be induced by the use of ribozymes or catalytic RNA. In this approach, the inhibitory nucleic acids would comprise either naturally occurring RNA (ribozymes) or synthetic nucleic acids with catalytic activity.

Inhibitory nucleic acids can also include aptamers, which are short, synthetic oligonucleotide sequences that bind to proteins (see, e.g., Li et al. (2006) Nuc. Acids Res. 34: 6416-24). They are notable for both high affinity and specificity for the targeted molecule, and have the additional advantage of being smaller than antibodies (usually less than 6 kD). Aptamers with a desired specificity are generally selected from a combinatorial library, and can be modified to reduce vulnerability to ribonucleases, using methods known in the art.

Peptide Inhibitors

VWF activity can be inhibited using peptide antagonists. For example, peptides comprising a subsequence of the full length VWF polypeptide, especially those within various domains of VWF of defined activity (e.g., the D′/D3, A1, A3, C1, and the “cysteine knot” domains). Such peptide subsequences have from about 10-20, 20-30, 30-40, 40-50, 50-60, 60-75, 75-100, 100-150, 150-200, or more amino acid residues. One of skill can derive an inhibitory peptide from human von Willebrand Factor, or from species orthologs, homologs, or variants of these sequences.

Peptide antagonists for VWF also include peptides that do not correspond to VWF sequences. For example, peptides selected from combinatorial libraries can serve to inhibit VWF activity.

Inactivating Antibodies

Inhibition of VWF activity can be achieved with an inactivating antibody. An inactivating antibody can comprise an antibody or antibody fragment that specifically binds to VWF. Inactivating antibody fragments include, e.g., Fab fragments, heavy or light chain variable regions, single complementary determining regions (CDRs), or combinations of CRDs with VWF binding specificity.

Any type of inactivating antibody can be used according to the methods of the invention. Generally, the antibodies used are monoclonal antibodies. Monoclonal antibodies can be generated by any method known in the art (e.g., using hybridomas, recombinant expression and/or phage display).

Antibodies can be derived from any appropriate organism, e.g., mouse, rat, rabbit, gibbon, goat, horse, sheep, etc. To reduce undesirable antigenicity, such an inactivating antibody can be a chimeric (e.g., mouse/ human) antibody comprising the variable regions of a murine antibody that specifically binds VWF and a human antibody constant regions, or a humanized antibody comprising the CDRs of a murine antibody that specifically binds VWF and a human antibody constant regions plus framework regions in the various regions. Furthermore, human antibodies can be made from human immune cells residing within an animal body.

### D. Identification of VWF Inhibitors

One can identify compounds that are therapeutically effective VWF inhibitors by screening a variety of compounds and mixtures of compounds for their ability to inhibit VWF activity, either by suppressing VWF expression or by interfering with VWF biological activity, e.g., to prevent VWF binding with other proteins. The testing can be performed using a minimal region or subsequence of VWF or a target protein, or a full length polypeptide.

An aspect of the present invention relates to methods for screening compounds for inhibiting VWF activity. Such compounds can be in substantially isolated form or as a mixture of multiple active ingredients. An example of an in vitro binding assay can comprise a VWF polypeptide or a fragment thereof; a test binding compound; and a protein or a fragment thereof that is known to bind VWF. Another example of binding assay comprises a mixture of synthetically produced or naturally occurring compounds, such as a cell culture broth. Suitable cells include any cultured cells such as mammalian, insect, microbial (e.g., bacterial, yeast, fungal) or plant cells.

In addition to assaying for an effect on VWF-target protein binding to identify suitable inhibitors, one can test directly for a compound's effect on infarction. Animal models for infarction, such as the middle cerebral artery (MCA) occlusion mouse model, are known in the art, and can be utilized to assess the efficacy of any test compound as a VWF inhibitor. The examples in this disclosure provide a detailed description of the MCA occlusion mouse model that can be used to verify the efficacy of a putative VWF inhibitor, for instance, following its identification in an in vitro binding assay.

In preferred embodiments, the screening assays for VWF inhibitors are designed to screen large chemical libraries by automating the assay steps and providing compounds from any convenient source to assays, which are typically run in parallel (e.g., in microtiter formats on microtiter plates in robotic assays). A high throughput format can be appropriate, particularly for the preliminary in vitro screening assays.

In some assays it will be desirable to have positive controls to ensure that the components of the assays are working properly. For example, a known VWF inhibitor (such as ADAMTS13) can be included in the assay, and the resulting effects on infarction can be determined according to the methods described herein.

Essentially any chemical compound can be tested as a potential VWF inhibitor for use in the methods of the invention. Most preferred are generally compounds that can be dissolved in aqueous or organic (especially DMSO-based) solutions are used. It will be appreciated that there are many suppliers of chemical compounds, such as Sigma (St. Louis, Mo.), Aldrich (St. Louis, Mo.), Sigma-Aldrich (St. Louis, Mo.), and Fluka Chemika-Biochemica Analytika (Buchs Switzerland).

Inhibitors of VWF activity or binding can be identified by screening a combinatorial library containing a large number of potential therapeutic compounds (potential modulator compounds). Such “combinatorial chemical libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

Alternatively, one can identify compounds that are suitable VWF inhibitors by screening a variety of compounds and mixtures of compounds for their ability to inhibit VWF expression. Methods of detecting expression levels are well known in the art, and include both protein- and nucleic acid-based methods.

For example, a test compound can be contacted in vitro with cells expressing VWF. An inhibitor that suppresses VWF expression is one that results in a decrease in the level of VWF polypeptide or transcript, as measured by any appropriate assay common in the art (e.g., Northern blot, RT-PCR, Western blot, or other hybridization or affinity assays), when compared to expression without the test compound. In some embodiments, a test nucleic acid inhibitor can be introduced into a cell, e.g., using standard transfection or transduction techniques, and the level of VWF expression detected.

The present invention will be further illustrated in the following examples, without any limitation thereto.

### Examples

### A. Materials and Methods

Mice

The Adamts13−/−, Vwf−/−, and Adamts13−/−/Vwf−/− mice described in this study were on C57BL/6J background. The control WT mice on C57BL/6J background were purchased from The Jackson Laboratory, Bar Harbor, Me. The mice used were 8-10 weeks old males. Animals were bred at the Immune Disease Institute, and experimental procedures were approved by its Animal Care and Use Committee.

Preparation of ADAMTS13 Protein

r-hu ADAMTS13 was expressed by stably transfected HEK293 or CHO cell lines in serum free medium. Following a volume reduction by ultradiafiltration, r-hu ADAMTS13 was purified by applying a conventional multi step chromatography. r-hu ADAMTS13 purified to homogeneity was characterized by SDS-PAGE under reducing and non-reducing conditions and Western blotting using a rabbit polyclonal anti ADAMTS13 antibody. The activity was assessed by the FRETS-VWF73 assay as described, e.g., in Kokame et al. (2005) Br. J Haematol. 129:93-100. r-hu ADAMTS13 protein was dissolved in 150 mmol NaCl/20 mmol Histidin/2% Sucrose/0.05% Crillet 4HP (Tween 80), pH 7.4 (Baxter Bioscience, Vienna, Austria). Control (vehicle) used in experiments was buffer in which r-hu ADAMTS13 was dissolved.

Middle Cerebral Artery Occlusion (MCAO) Stroke Model

Transient focal cerebral ischemia was induced by 2 hours occlusion of the right middle cerebral artery with a 7.0 siliconized filament in male mice. We checked by black ink infusion that the architecture of blood vessels in the middle cerebral artery region did not show any obvious differences among the mouse genotypes used in this study. Mice were anesthetized with 1-1.5% isoflurane in 30% oxygen. Body temperature was maintained at 37° C.±1.0 using a heating pad. Laser Doppler flowmetry was used in all mice to confirm induction of ischemia and reperfusion. At 10 minutes before reperfusion (110 minutes after MCAO), r-hu ADAMTS13 (3460 U/kg, Baxter Bioscience, Vienna, Austria) or vehicle was injected intravenously. At 22 hours after MCAO, mice were sacrificed. Eight 1 mm coronal sections were stained with 2% triphenyl-2,3,4-tetrazolium-chloride (TTC). Sections were digitalized and infarct areas were measured blindly using the NIH Image software.

Tape Removal Test

Mice were subjected to 1 hour of MCAO. They were injected with r-hu ADAMTS13 (derived from CHO cell, 3460 U/kg, Baxter Bioscience, Vienna, Austria) or vehicle 10 minutes before reperfusion (50 minutes after MCAO) and were tested 24 hours post-surgery. The tape removal test allows the assessment of sensory and motor impairments in forepaw function and was adapted from previous studies in rats (Zhao et al. (2006) Nat. Med. 12:441-45). Mice were held and 6 mm diameter round tapes were placed onto the plantar surface of the two forepaws so that they covered the hairless part of the forepaws. The animal was then placed in a box (40 cm×30 cm) and the times the animal took to remove the pieces of tape from the ipsilateral and contralateral paws were recorded. The animals were given a maximum of 180 seconds to sense the tapes and then remove them and were scored as 180 seconds if they did not succeed.

Measurement of Plasma IL-6 Levels

Blood samples were obtained 22 hours after 2 hours of MCAO by retro-orbital bleeding into tubes containing 30 U/mL Enoxaparin (Aventis Pharmaceutical Products, Bridgewater, N.J.) in phosphate-buffered saline (PBS). Plasma was separated by centrifugation. IL-6 protein concentration was measured by ELISA (R&D Systems, Minneapolis, Minn.) according to the manufacturer's guidelines.

Quantification of Neutrophils

Twenty-two hours after MCAO (2 hours), mice were sacrificed by overdose of isofluorane, perfused with ice-cold PBS (pH 7.4) and brains were harvested. Brain cryosections (20 μm) were stained with H&E and the extra vascular neutrophils were counted blindly in the peri-infarct areas using a light microscope at 40× magnification. For each animal, 3 fields in 3 sections (2 mm apart) from the ischemic hemisphere were analyzed. Values represent the number of neutrophils per mm². Three animals were evaluated per group.

Bleeding Time

Mice (8-9 weeks old) were anesthetized with 2.5% Avertin (15 μl/g mouse body weight, IP) and a 3 mm segment of tail was amputated. The tail was immersed in phosphate buffer saline at 37° C., and the time required for the stream of blood to stop for more than 30 seconds was defined as the bleeding time.

Statistical Analysis

Results are reported as the mean±S.E.M. Statistical comparisons were performed using ANOVA followed by Fisher's PLSD test or Boneferroni's multiple comparison test. P<0.05 was considered significant. For IL-6 measurement in plasma, the statistical significance was assayed using the Kruskal-Wallis nonparametric test followed by the Dunn's multiple comparison test. P<0.05 was considered significant.

### B. Example 1

### Deficiency in VWF Reduces Infarct Volume in the Intraluminal MCAO Model in Mice

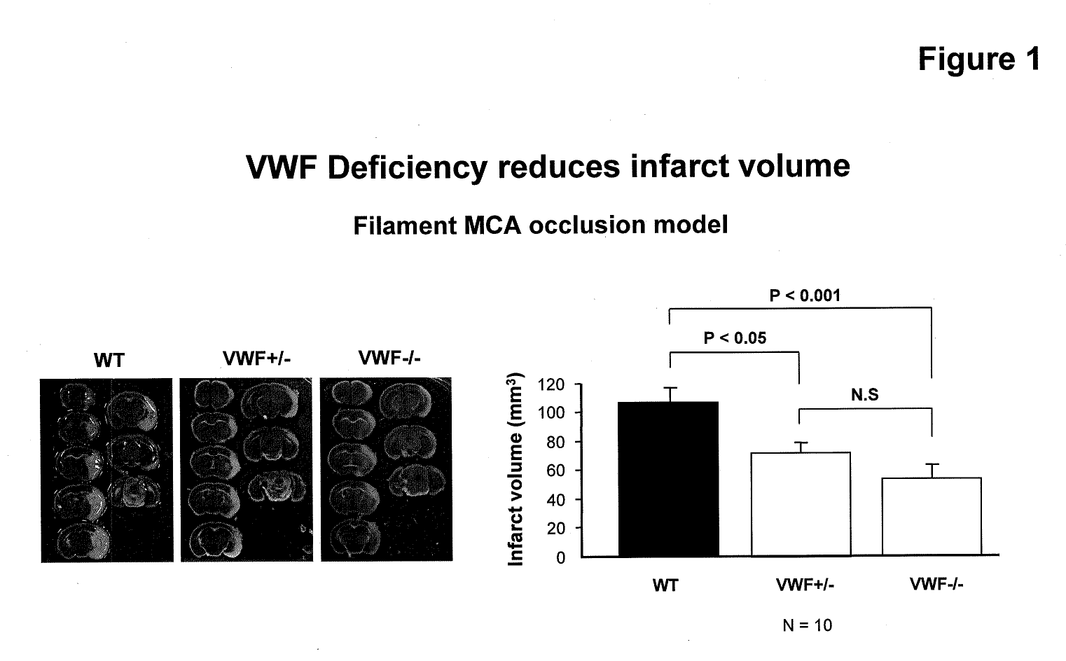

Transient occlusion of the right middle cerebral artery (MCA) was achieved by a monofilament insertion up to the MCA. After 2 hours, the monofilament was withdrawn to allow reperfusion. Infarct volume was measured by 2% 2,3,5-triphenyltetrazolium hydrochloride (TTC) staining at 24 h after cerebral ischemia (FIG. 1). Data are expressed as mean±SEM (n=10).

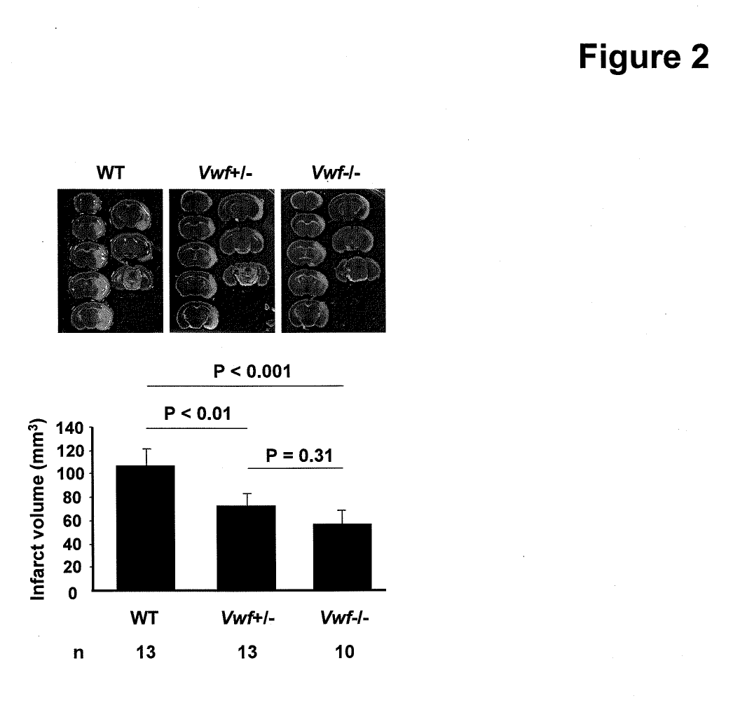

In a follow up test to address the importance of VWF levels in stroke outcome, we subjected wild-type (WT), Vwf± and Vwf−/− mice to 2 hours of focal cerebral ischemia using the MCAO stroke model, and examined mouse brains 22 hours later using triphenyl-2,3,4-tetrazolium-chloride (TTC) staining to quantify infarct size (FIG. 2). We observed that deficiency in VWF caused a two-fold reduction in infarct volume compared to WT (P<0.05). In the Vwf± mice the infarct volume was reduced by nearly 40% (P<0.05, FIG. 2), showing that decreasing VWF to 50% is sufficient to drastically reduce stroke impact (Denis et al. (1998) Proc Natl Acad Sci USA 95:9524-29).

The results show that deficiency of VWF dramatically reduces infarct volume 22 hours after cerebral ischemia. Surprisingly, VWF heterozygosity also significantly reduced infarct size, which we confirmed in a second double blinded study. VWF haploinsuffiency, not detected in previous studies of these mice, shows the importance of VWF level in thrombosis, in particular, in the brain. For example, ferric chloride did not induce thrombosis in mesentery arterioles in heterozygotes. The results are promising for improving the outcome of cerebral infarction with even a partial reduction of VWF induced clotting activity.

### C. Example 2

### Recombinant Human VWF Increases Infarct Volume

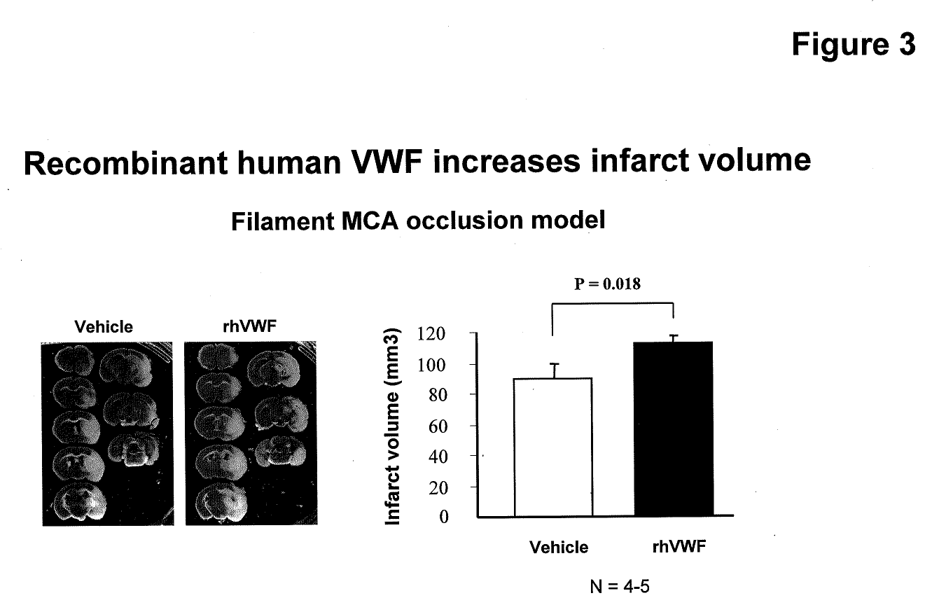

Mice were subjected to 2 h transient focal ischemia. Recombinant human VWF (0.8 mg/kg body weight) was infused 10 min before reperfusion and repeated 3 h later. Treatment with rhVWF increased infarct volume 24 h after stroke compared with vehicle-treated control group (FIG. 3). Data are expressed as mean±SEM (n=4-5).

### D. Example 3

### ADAMTS13 Negatively Regulates Infarction after Cerebral Ischemia

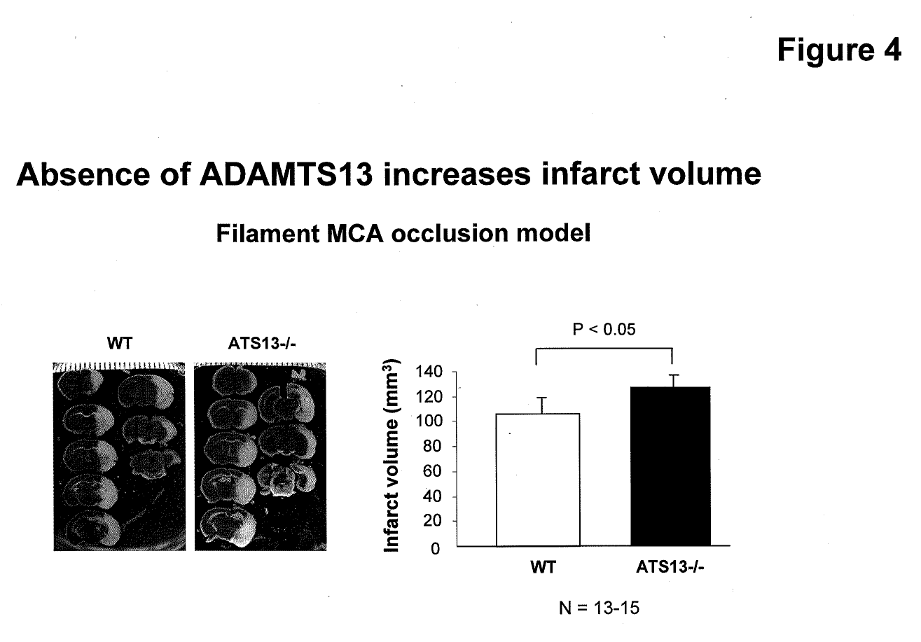

Mice were subjected to 2 h transient focal ischemia and infarct volume was measured 24 h after stroke (FIG. 4). Data are expressed as mean±SEM (n=13-15).

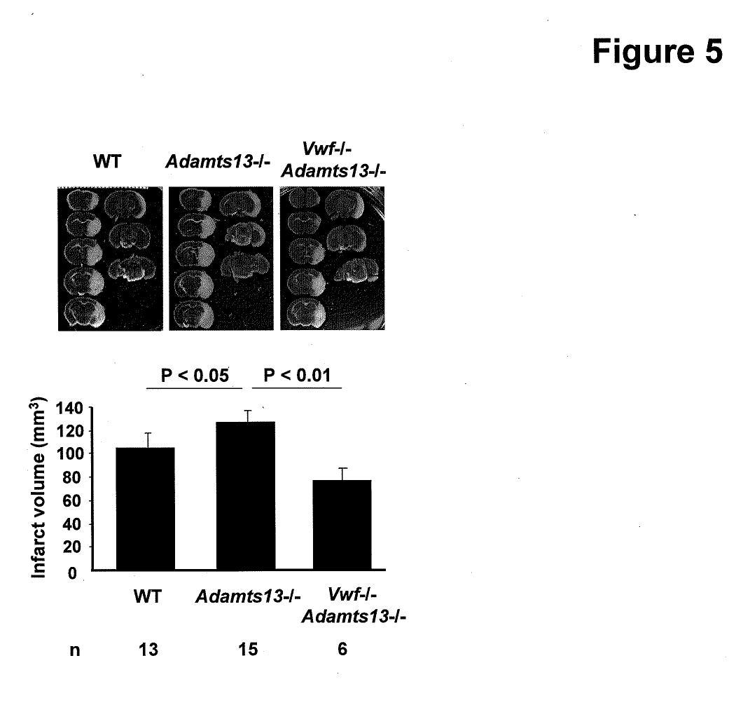

We ran a follow up test to evaluate the protective role of ADAMTS13 in ischemic stroke. Indeed, Adamts13−/− mice showed significantly increased infarct volume after MCAO compared to WT mice (124.12±6.59 vs. 103.65±6.69, P<0.05, FIG. 5). The function of ADAMTS13 in stroke was dependent on its action on VWF, because mice deficient in both ADAMTS13 and VWF had infarct volume similar to mice deficient in VWF alone (P=0.28, FIGS. 1, 5).

We next compared the inflammatory response of WT and Adamts13−/− mice to stroke. At 22 hours after the MCAO, we did not observe differences in neutrophil recruitment to the peri-infarct region as determined by counting the neutrophils in H&E-stained brain sections (WT 36±4, Adamts13−/−40±9 per mm²; not significant). Within the infarct, neutrophil counts were lower though similar in these two groups. We measured plasma levels of IL-6, an indication of peripheral immune system activation, at 22 hours after 2 hours MCAO. Compared with sham-operated mice, we confirmed a significant elevation of IL-6 in the plasma of mice that underwent MCAO (Table 1). However, there was no difference in plasma levels of IL-6 between WT and Adamts13−/− mice after MCAO surgery. Therefore, it is unlikely that the larger infarcts observed in the Adamts13−/− are a result of an enhanced neutrophil infiltration in these mice.

TABLE 1
Plasma levels of IL-6 in wild type and ADAMTS13−/− mice
22 hours after ischemia

| | | | Plasma IL-6 |
|---|---|---|---|
| Mouse | Treatment | n | (pg/ml) |
| Wild type | Sham | 10 | 42.2 ± 11.3 |
| Wild type | MCAO | 15 | 252.8 ± 82.2 |
| ADAMTS13−/− | MCAO | 10 | 242.9 ± 67.7 |

### E. Example 4

### Recombinant Human ADAMTS13 Reduces Infarct Volume and Improves Stroke Outcome after Cerebral Ischemia

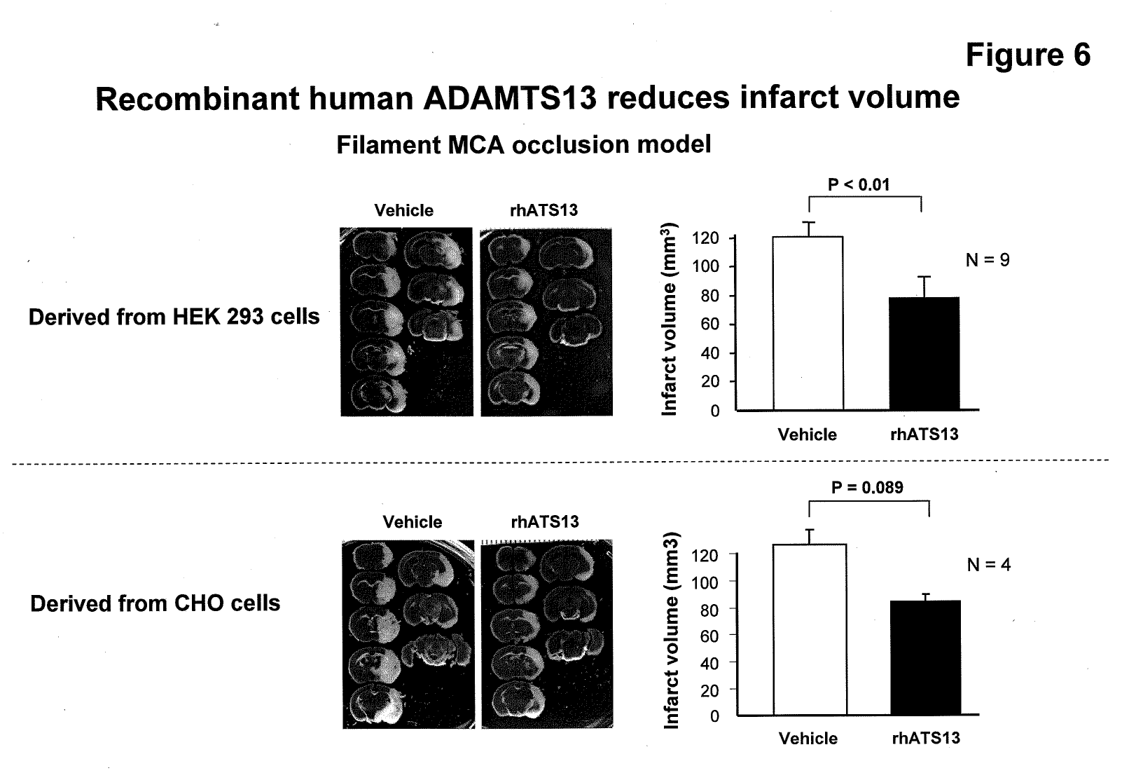

Mice were subjected to 2 h transient focal ischemia and infarct volume was measured 24 h after stroke. Recombinant human ADAMTS13 (3258 U/kg body weight) was infused 10 min before reperfusion. Results are shown in FIG. 6. Compared with the vehicle-treated group, administration of rhADAMTS13 derived from HEK293 cells significantly reduced infarct volume (n=9). Treatment with rhADAMTS13 derived from CHO cells also resulted in a reduction in infarct volume (FIG. 6). Data are expressed as mean±SEM (n=4).

We have shown that endogenous ADAMTS13 reduces infarct volume after ischemic stroke. In a follow up study, we evaluated the therapeutic potential of infusion of additional recombinant human ADAMTS13 (r-hu ADAMTS13) into WT mice. To emulate clinical situations, we infused the protein 110 minutes after ischemic occlusion, i. e., just prior to removing the blocking filament resulting in reperfusion. During the period of stasis, thrombi form in the artery as this MCAO stroke model is highly dependent on platelets and their adhesion receptors including the receptors for VWF, β3 integrin and GPIbα.

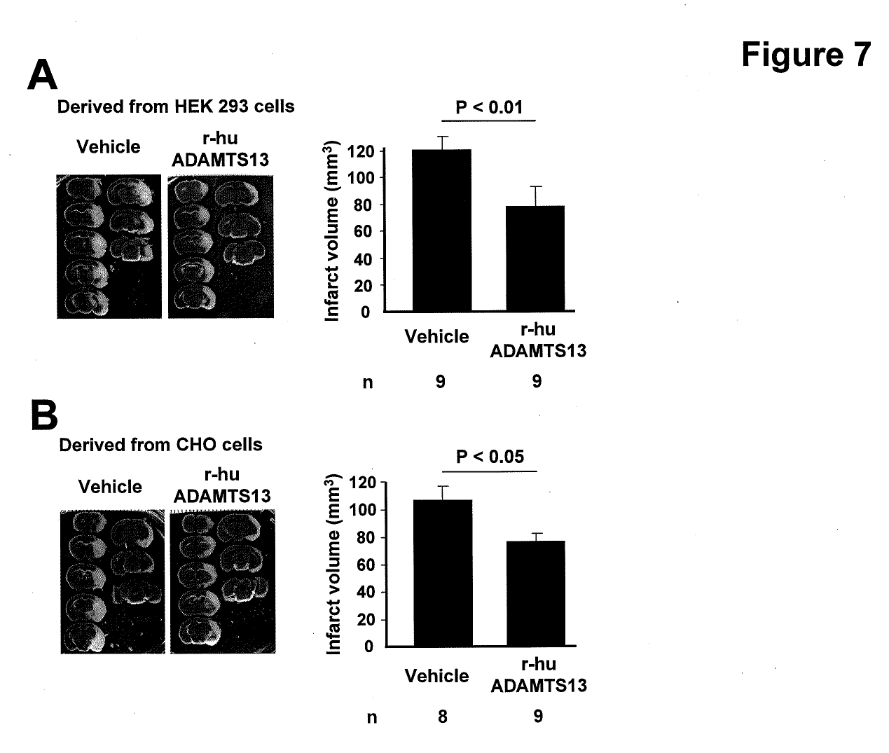

We prepared r-hu ADAMTS13 in two different cell lines (HEK 293 and CHO cells), to account for possible differences in glycosylation. Indeed there were differences in the glycosylation pattern resulting in a different half life of the two preparations in mouse circulation (HEK 293 ADAMTS13<1 hour and CHO cell ADAMTS13 several hours). We have previously shown that r-hu ADAMTS13 prepared in HEK 293 reduces platelet plug size in the ferric chloride arterial injury model in mice (Chauhan et al. (2006) J. Exp. Med. 203:767-76). The r-hu ADAMTS13 cleaved both mouse and human VWF with similar efficiency. Despite the differences in half life, at the high concentration infused, both of the r-hu ADAMTS13 preparations were similarly effective, reducing infarct volume by approximately 30% (FIGS. 7A, B).

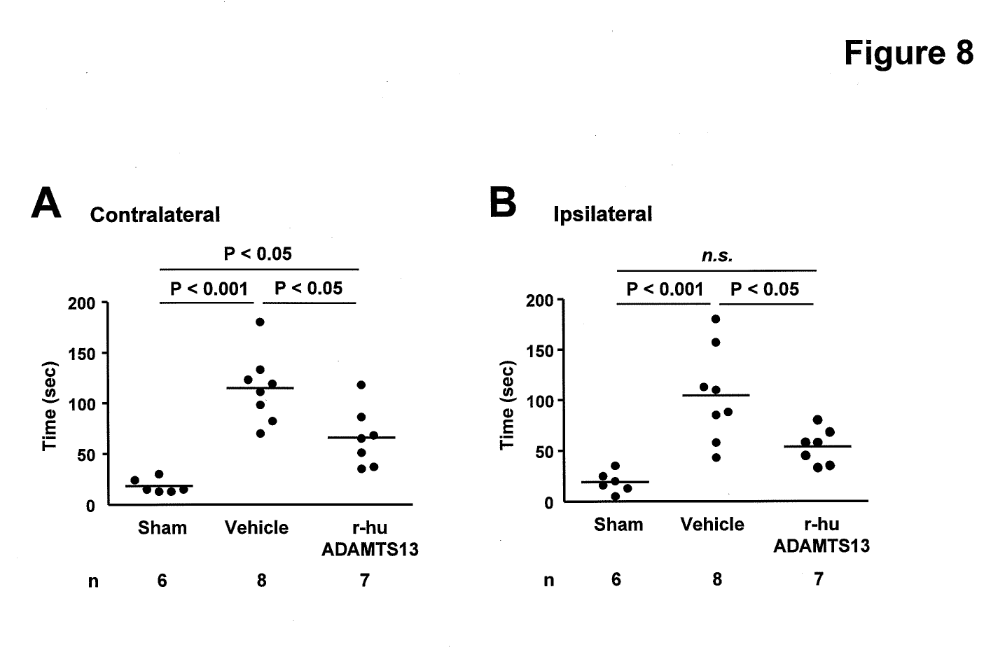

To test whether the reduction in infarct volume actually improves functional outcome, we performed the tape removal test, a technique that assesses sensory and motor impairments in forepaw function (Bouet et al. (2007) Exp. Neurol. 203:555-67). Twenty four hours after surgery, mice that underwent one hour MCAO showed an increase in the time needed to remove adhesive tape from the contralateral and ipsilateral paws compared to sham-operated mice (FIG. 8), consistent with previous reports. Interestingly, treatment with r-hu ADAMTS13 (CHO-cell derived) significantly shortened the time to remove the adhesive tape from either paw when compared to vehicle treated mice (P<0.05), indicating a profound improvement in sensorimotor performance of the r-hu ADAMTS13 treated mice. Taken together, these results show a protective effect of r-hu ADAMTS13 when infused after cerebral ischemia.

Based on the observation that VWF levels modulate infarction; it could be hypothesized that the outcome of stroke would be worse in individuals with high VWF. Plasma VWF levels vary over a wide range in humans. ADAMTS13 regulates VWF activity, not by decreasing VWF levels, but by cleaving the UL-VWF into smaller less adhesive multimers (i.e., reducing VWF activity, as defined herein). ADAMT13 deficiency increased infarct size after cerebral ischemia, indicating the importance of VWF size (as opposed to absolute levels) on stroke outcome. r-hu ADAMTS13 prepared in two different cell lines significantly reduced infarct volume when infused 110 min after cerebral ischemia, indicating that r-hu ADAMTS13 infusion after an ischemic event diminishes the deleterious consequences. Surprisingly, infusion of r-hu ADAMTS13 significantly improved the sensorimotor performance of mice in a test shown to be useful in evaluating outcome of ischemia produced by MCAO in the mouse.

### F. Example 5

### ADAMTS13 Infusion Improves Hemostatic Function of Mice with Cerebral Ischemia

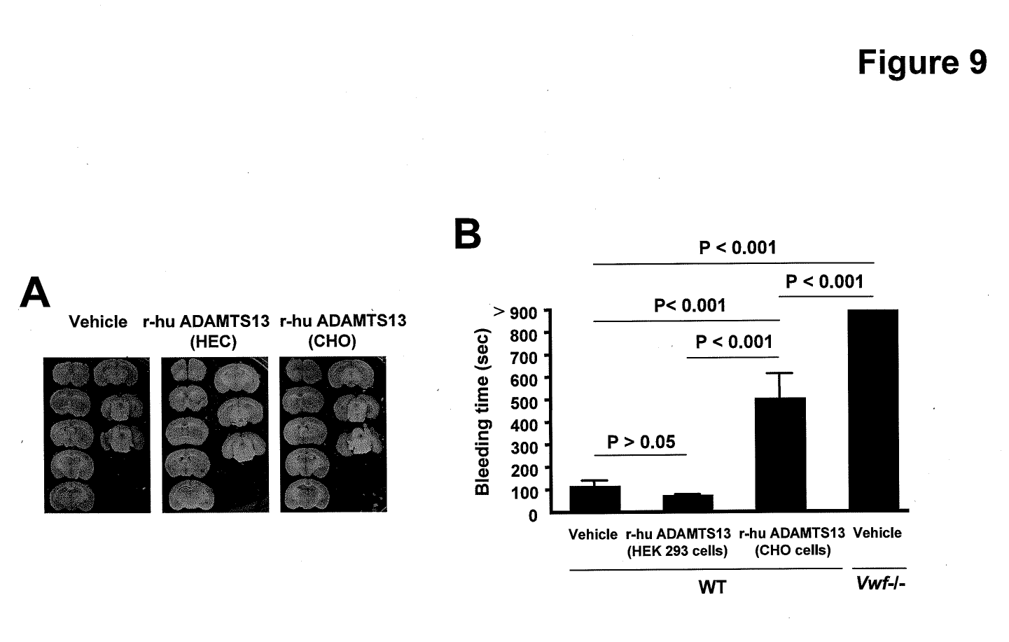

Cerebral hemorrhage was not observed in any WT mice treated with either r-hu ADAMTS13 preparation (FIG. 9A). Interestingly, we also did not detect cerebral hemorrhage in Vwf−/− or Vwf± mice. We have previously reported that platelet depletion in this MCAO model causes significant bleeding in the affected hemisphere. Thus, the role of platelets in prevention of hemorrhage at stroke sites is preserved in VWF-deficiency and after r-hu ADAMTS13 treatment.

To examine to what extent r-hu ADAMTS13impacts hemostasis in the periphery, we also measured tail bleeding time in WT mice 5 hours after infusion with r-hu ADAMTS13 and compared to mice treated with vehicle and to Vwf−/− mice. Vwf−/− mice had a highly prolonged bleeding time (FIG. 9B), with all of the animals requiring cauterization, confirming on a pure background the severe bleeding phenotype of these mice. The HEK 293 preparation with short half life of r-hu ADAMTS13 did not affect bleeding, while the CHO cell preparation with long half life prolonged bleeding time but to a lesser extent than VWF deficiency (FIG. 9B). Reduction of VWF multimer size by ADAMTS13 had a less drastic effect on bleeding than VWF deficiency because the shorter VWF species retained some hemostatic activity.

ADAMTS13 dismantles existing thrombi and prevents new thrombi from forming by cleaving the VWF multimers present in the thrombus and the UL-VWF released locally from Weibel-Palade bodies. Furthermore, as demonstrated herein, neither of the r-hu ADAMTS13 preparations produced cerebral hemorrhage in any of the treated brains. In contrast, tPA induces gross cerebral hemorrhage at 24 h in the MCAO model, as does blockade of the platelet integrin receptor αIIbβ3 (Kleinschnitz et al. (2007) Circulation 115:2323-30; Cheng et al. (2006) Nat. Med. 12:1278-85). Interestingly, the ADAMTS13 preparation with short half life was equally effective in reducing infarct volume without affecting bleeding time. Taken together, the results indicate that treatment of ischemic stroke with r-hu ADAMTS13 is safer than tPA or αIIbβ3 blockade.

It is understood that the examples and embodiments described herein are for illustrative purposes only and that various modifications and changes in light thereof will be suggested to persons skilled in the art and are to be included within the purview of this application and are considered to be within the scope of the appended claims. All patents, patent applications, and other publications cited in this application, including published amino acid or polynucleotide sequences, are incorporated by reference in the entirety for all purposes.

What is claimed is:

1. A method for treating or preventing an infarction in an individual, comprising the step of administering to the individual a pharmaceutical composition comprising a therapeutically effective amount of ADAMTS13 protein or a biologically active derivative thereof, thereby treating or preventing infarction in the individual.

2. The method of claim 1, wherein the infarction occurs in the brain,.

3. The method of claim 1, wherein said administration does not affect a peripheral immune response.

4. The method of claim 1, wherein the ADAMTS13 protein is glycosylated.

5. The method of claim 1, wherein the ADAMTS13 protein has a plasma half-life of more than 1 hour.

6. The method of claim 1, wherein the ADAMTS13 protein is recombinantly produced by HEK293 cells.

7. The method of claim 1, wherein the ADAMTS13 protein is recombinantly produced by CHO cells.

8. The method of claim 1, wherein the pharmaceutical composition is administered multiple times or by continuous infusion.

9. The method of claim 1, wherein the pharmaceutical composition is administered within 110 minutes of detection of the infarction.

10. The method of claim 1, further comprising a step of determining the level of VWF in the individual.

11. The method of claim 10, wherein the amount of said ADAMTS13 or biologically active derivative thereof is determined based on the plasma level of VWF in the individual.

12. The method of claim 1, wherein said administration does not increase the level of hemorrhage, as compared to the level of hemorrhage in an individual not receiving the pharmaceutical composition.

13. The method of claim 1, wherein said administration reduces infarct volume 22 hours after administration.

14. A method of improving the recovery of sensorimotor function in an individual that has experienced a cerebral infarction, comprising the step of administering to the individual a pharmaceutical composition comprising a therapeutically effective amount of ADAMTS13 protein or a biologically active derivative thereof, thereby improving the recovery of sensorimotor function in the individual.

15. Use of a pharmaceutically effective amount of ADAMTS13 protein or a biologically active derivative thereof for the preparation of a pharmaceutical composition for treating or preventing an infarction.

16. The use of claim 15, wherein the infarction occurs in the brain.

17. The use of claim 15, wherein the ADAMTS13 protein is recombinantly produced by HEK293 cells.

18. The use of claim 17, wherein the ADAMTS13 protein is recombinantly produced by CHO cells.
